(19) United States
(12) Patent Application Publication
(10) Pub. No.: US 2008/0037851 A1
(43) Pub. Date: Feb. 14, 2008

(54) MEDICAL IMAGE SYNTHESIS METHOD AND APPARATUS

(76) Inventor: Takuzo TAKAYAMA, Otawara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/835,681

(22) Filed: Aug. 8, 2007

(30) Foreign Application Priority Data
Aug. 9, 2006 (JP) 2006-217145

(51) Int. Cl. A61B 5/00

(52) U.S. Cl. 382/131

(57) ABSTRACT

There is disclosed a medical image synthesis method which comprises acquiring a plurality of different functional images in which functions of a test subject are imaged, taking a logical product of these functional images to generate new information, and superimposing and displaying the new information on a corresponding position of the functional image of the test subject.

### CROSS-REFERENCE TO RELATED APPLICATIONS

This application is based upon and claims the benefit of priority from prior Japanese Patent Application No. 2006-217145, filed Aug. 9, 2006, the entire contents of which are incorporated herein by reference.

### BACKGROUND OF THE INVENTION

1. Field of the Invention

The present invention relates to, for example, a nuclear medicine device, a magnetic resonance imaging device, an ultrasonic diagnosis device or the like. More particularly, it relates to a medical image synthesis method which acquires a functional image by imaging functions such as metabolism and life and death of cells in a human body as a test subject, and a medical image synthesis apparatus.

2. Description of the Related Art

A medical image includes a morphological image and a functional image. The morphological image is an image for determining, for example, shape, size, movement and the like of a bone, a tumor and the like of a human body as a test subject. Examples of the morphological image include a computed tomography (CT) image acquired by an X-ray imaging device (X-ray CT device), and an X-ray image acquired by an X-ray device. Furthermore, examples of the morphological image include a morphological image acquired by a magnetic resonance imaging (MRI) device and an ultrasonic image acquired by an ultrasonic (US) diagnosis device.

The functional image is an image for knowing functions such as metabolism and circulation in the body. To obtain the functional image, for example, in a nuclear medicine device, a behavior of a radioactive marker administered to the body is tracked with an elapse of time, and functions such as the metabolism and circulation are imaged. The functional image is acquired by nuclear medicine devices such as a single photon emission computed tomography (SPECT) imaging device and a positron emission computed tomography (PET) imaging device. Furthermore, examples of the functional image include the diffusion image acquired by the MRI device and the ultrasonic image acquired by the US device.

As an image capture technique of the functional image, fluorodeoxyglucose (FDG) imaging is performed using the PET device. FDG is an 18F radioactive marker for the PET device which is used to inspect a tumor in the body. FDG imaging is used to image a cancerous region by utilizing the fact that more glucose is taken into the cancerous region than into a normal region.

On the other hand, diffusion imaging acquired by the MRI device is also known as a functional image capable of imaging the cancerous region. In diffusion imaging, a phase of an MRI signal slightly changes by micro-movements (diffusions) of water molecules. In consequence, the diffusion imaging is a technique of capturing and imaging the phase change of the MRI signal by an imaging sequence called a motion probing gradient (MPG) pulse. The MPG pulse is an additional gradient magnetic pulse to emphasize a phase deviation of a transverse magnetization spin by micro-movements of molecules including target nuclear species of image pixels.

Examples of an image capture process for use in the MRI device include a spin echo (SE) process and an echo planar imaging (EPI) process. In these image capture processes, when a repeating time and an echo time are set to be appropriate, a density of the target nuclear species themselves and the like can be reflected on a signal value of the image. Furthermore, in these image capture processes, the additional gradient magnetic pulse (MPG pulse) is applied during a time from application of an excitation pulse to collection of echo data, whereby specific information can be given to the image. The MPG pulse in the MRI device is disclosed in, for example, Jpn. Pat. No. 3,679,892.

The diffusion of the micro-movements of the water molecules, for example, decreases in an infarction region and increases in a tumor region as compared with a normal region. In the diffusion imaging, therefore, the normal region, the infarction region, the tumor region and the like are distinguished from one another in accordance with a degree of the diffusion. In the image capture for diffusion imaging, an ultra-high-speed imaging process which is scarcely influenced by physiologic movements such as body movement, respiration and heartbeat is used as an assumption. Moreover, in the image capture for the diffusion imaging, a multi-coil called a phased array coil is used in order to improve the signal-to-noise ratio of the image.

In recent years, for body diffusion weighted imaging, the image capture for the diffusion imaging is performed many times, and the images acquired by the image capture are added and averaged, so that the signal-to-noise ratio of the image is further improved.

FDG imaging by means of the PET device is utilized for imaging the cancerous region. It is known that glucose such as FDG for use in the FDG imaging is accumulated in not only the cancerous region but also the liver, brain, heart after eating, kidney, bladder, digestive canal, glandular system and inflammation region in a physiologic accumulation order.

Therefore, in FDG imaging, not only the cancerous region but also regions other than the cancerous region, for example, liver regions such as hemangioma, abscess and intrahepatic angiocholitis, and further regions influenced by the heartbeat are also imaged with high possibility. That is, FDG imaging is used for imaging the cancerous region, but there is a very low possibility that the cancerous region is securely captured. As a result, in a case where the image acquired by the FDG imaging is read to perform diagnosis, it is very difficult to judge whether a target is the cancerous region or a region other than the cancerous region.

Diffusion imaging by means of the MRI device is also known as the functional image capable of imaging the cancerous region. In diffusion imaging, however, for example, the normal region, the infarction region, the tumor region and the like are distinguished from one another in accordance with the degree of diffusion. Therefore, in diffusion imaging, there is a high possibility of imaging the regions other than the cancerous region, for example, liver regions such as hemangioma, abscess and intrahepatic angiocholitis, and further regions influenced by the heartbeat, in the same manner as in FDG imaging by means of the PET device. Therefore, even in a case where the diagnosis is performed by the diffusion imaging, it is very difficult to judge whether the target is the cancerous region or the region other than the cancerous region.

An object of the present invention is to provide a medical image synthesis method in which the imaging of a functional image can be performed so that a cancerous region can highly precisely be estimated as compared with a case where the cancerous region is estimated by using a single functional image, and a medical image synthesis apparatus.

### BRIEF SUMMARY OF THE INVENTION

A first aspect of the present invention is directed to a medical image synthesis method comprising: acquiring a plurality of different functional images in which functions of a test subject are imaged; taking a logical product of the plurality of different functional images to generate new information; and superimposing and displaying the new information on a corresponding position of the functional image of the test subject.

A second aspect of the present invention is directed to a medical image synthesis method comprising: acquiring a plurality of different functional images in which functions of a test subject are imaged; and taking a logical product of the plurality of different functional images to generate a new functional image.

A third aspect of the present invention is directed to a medical image synthesis apparatus comprising: an image fetching section which fetches a plurality of different functional images acquired by a plurality of medical image devices, respectively; a generating section which takes a logical product of the plurality of different functional images to generate new information; a display; and a displaying section which superimposes the new information on a corresponding position of the functional image of a test subject to display the information in the display.

A fourth aspect of the present invention is directed to a medical image synthesis apparatus comprising: an image generation unit which fetches a plurality of different functional images acquired by a plurality of medical image devices, respectively, and which takes a logical product of the plurality of different functional images to generate a new functional image.

### BRIEF DESCRIPTION OF THE SEVERAL VIEWS OF THE DRAWING

FIG. 1 is a functional block diagram showing a first embodiment of a medical image synthesis apparatus according to the present invention;

FIG. 2 is a schematic constitutional diagram of a PET device for use in the apparatus;

FIG. 3 is a schematic view showing one example of a PET tomographic image acquired by the PET device for use in the apparatus;

FIG. 4 is a schematic view showing one example of a PET axial transverse image to be acquired by the PET device for use in the apparatus;

FIG. 5 is a schematic constitutional diagram of an MRI device for use in the apparatus;

FIG. 6 is a schematic view showing one example of an MRI tomographic image acquired by the MRI device for use in the apparatus;

FIG. 7 is a schematic view showing one example of an MRI axial transverse image acquired by the MRI device for use in the apparatus;

FIG. 8 is a diagram showing examples of count values with respect to positions of the PET axial transverse image and an MRI diffusion axial transverse image acquired by the apparatus;

FIG. 9 is a diagram showing examples of count values with respect to positions of the PET axial transverse image and the MRI diffusion axial transverse image obtained by subtracting threshold values acquired in a subtracting section of the apparatus;

FIG. 10 is a diagram showing one example of a count value of an AND image acquired by a generating section of the apparatus;

FIG. 11 is a schematic view showing one example of the AND image generated by the generating section of the apparatus;

FIG. 12 is a schematic view showing one example of an image obtained by superimposing the AND image generated by a synthesis section of the apparatus on a morphological image acquired by the MRI device;

FIG. 13 is a diagram showing display examples of images to be displayed in a display screen of a display of the apparatus;

FIG. 14 is a medical image synthesis flow chart of the apparatus;

FIG. 15 is a schematic view showing one example of a first PET image acquired by the PET device for use in the apparatus;

FIG. 16 is a schematic view showing one example of a second PET image acquired by the PET device for use in the apparatus;

FIG. 17 is a schematic view showing one example of an MRI and diffusion image acquired by the MRI device for use in the apparatus;

FIG. 18 is a schematic view showing a function of generating a new functional image by the synthesis section of the apparatus;

FIG. 19 is a schematic view of the whole new functional image acquired by the apparatus; and

FIG. 20 is a schematic view showing a superimposed image obtained by superimposing the new functional image on the MRI morphological image acquired by the apparatus.

### DETAILED DESCRIPTION OF THE INVENTION

A first embodiment of the present invention will hereinafter be described with reference to the drawings.

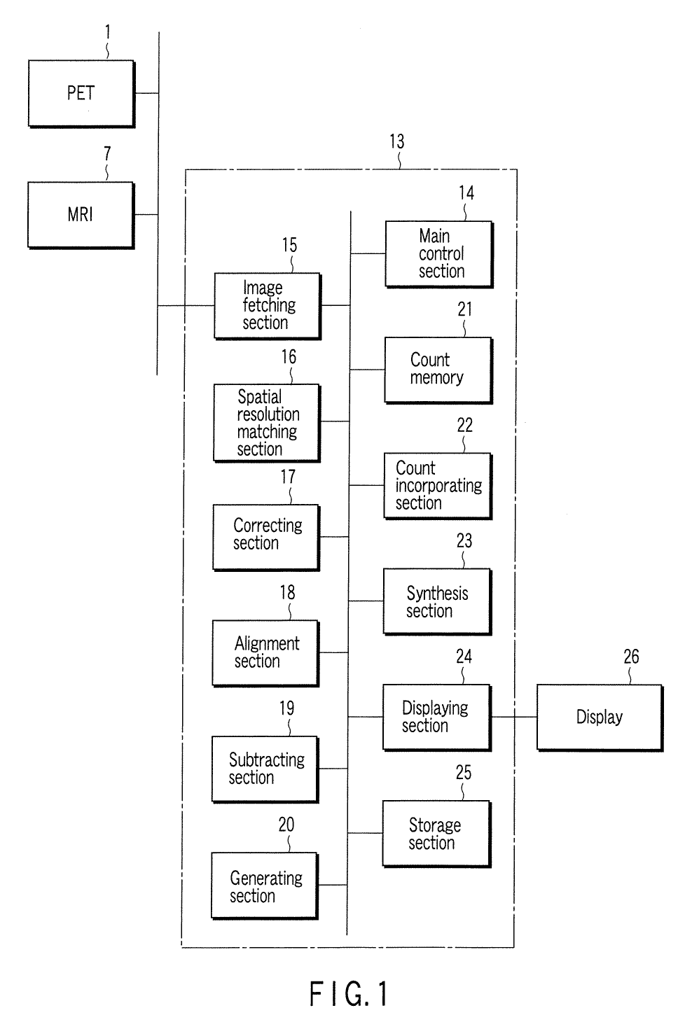

FIG. 1 is a functional block diagram of a medical image synthesis apparatus. In a PET device 1, a behavior at a time when a radioactive FDG marker of, for example, nuclear species 18F is administered to a human body as a test subject is tracked. In this case, by use of, for example, a fact that more glucose is taken in a cancerous region than in a normal region, the cancerous region is imaged to acquire a functional image.

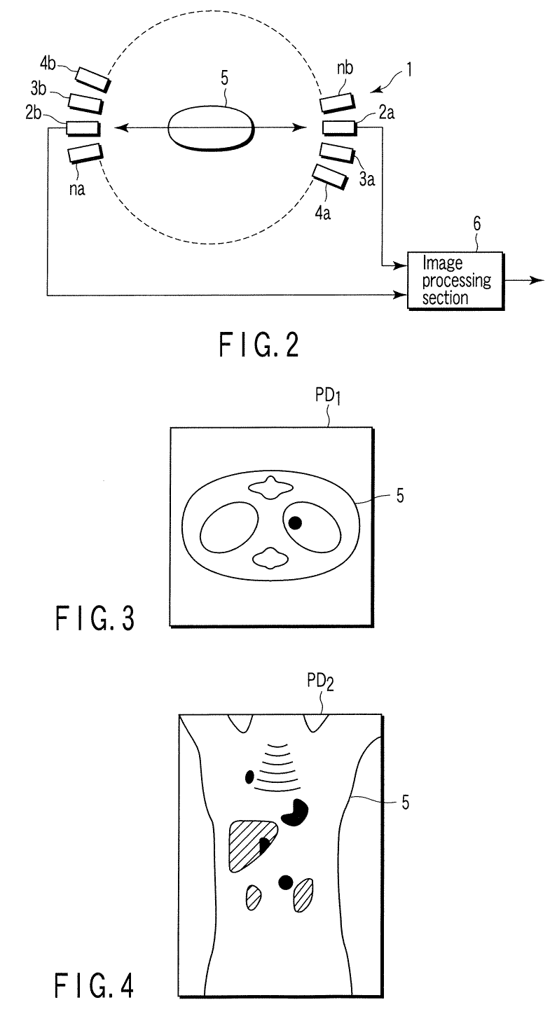

FIG. 2 shows a schematic constitutional diagram of the PET device 1. In the PET device 1, a plurality of pairs of detectors 2a and 2b, 3a and 3b, . . . , na and nb are arranged in a circular form. When the radioactive FDG marker is administered to a test subject 5 and gamma rays emitted from the positron nuclear species ¹⁸F in 180-degree opposite directions are simultaneously detected by the pair of detectors 2a and 2b, 3a and 3b, . . . , or n and nb, one count is output. For example, when the gamma rays are simultaneously detected by the pair of detectors 2a and 2b, the pair of detectors 2a and 2b output one count at this time.

An image processing section 6 fetches the count outputs of the pairs of detectors 2a and 2b, 3a and 3b, . . . , n and nb to output a distribution of FDG in the test subject 5 as a tomographic image (hereinafter referred to as the PET tomographic image). FIG. 3 shows one example of a PET tomographic image PD₁ of the test subject 5. When the PET device 1 scans the whole test subject 5, the image processing section 6 reconstructs an image from the tomographic images of the test subject 5 to output an axial transverse image (hereinafter referred to as the PET axial transverse image) PD₂ shown in, for example, FIG. 4.

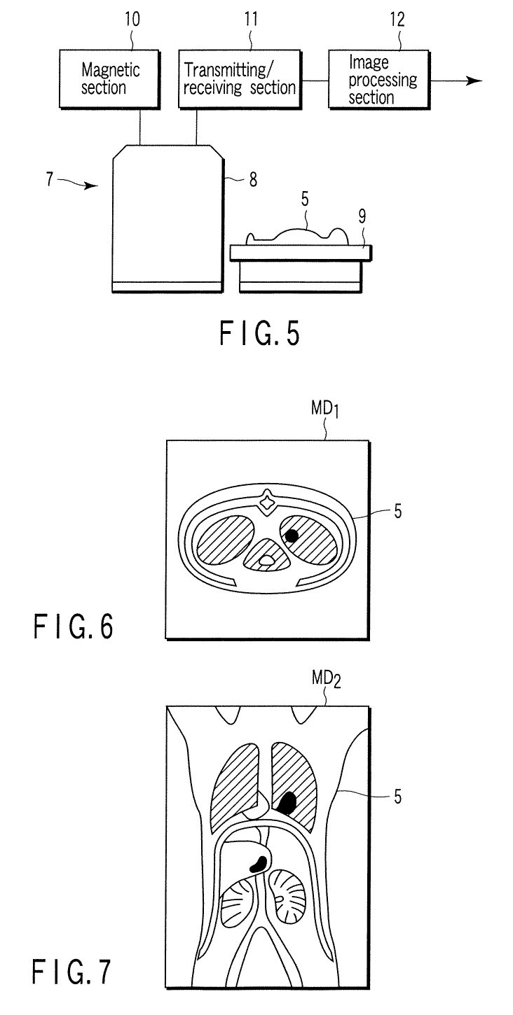

An MRI device 7 forms an image (hereinafter referred to as the diffusion image) of the cancerous region of the test subject 5 by diffusion imaging to output the image. FIG. 5 shows a schematic constitutional diagram of the MRI device 7. The MRI device 7 has a casing 8 for generation of a magnetic field and a bed 9. The test subject 5 is mounted on the bed 9. The casing 8 for generation of the magnetic field has, for example, a superconductive magnet and a gradient magnetic coil. The superconductive magnet generates a static magnetic field. The gradient magnetic coil generates a gradient magnetic field. A magnetic section 10 generates the static magnetic field and the gradient magnetic field from the superconductive magnet and the gradient magnetic coil to operate the fields, respectively. The casing 8 for generation of the magnetic field is provided with an RF coil. A transmitting/receiving section 11 transmits a high-frequency signal to the RF coil, and generates a high-frequency magnetic field from the RF coil to apply the field to the test subject 5. The transmitting/receiving section 11 detects, via the RF coil, a weak resonance signal at a time when the high-frequency magnetic field is applied to the test subject 5 to output an MRI signal.

In the MRI device 7, a phase of the MRI signal slightly changes with micro-movements (diffusions) of water molecules. In the diffusion imaging, the phase change of the MRI signal is captured by an imaging sequence in which an MPG pulse (an additional gradient magnetic pulse) is applied. An image processing section 12 subjects the MRI signal during the diffusion imaging to two-dimensional/three-dimensional Fourier transform to reconstruct an MRI diffusion image. FIG. 6 shows one example of an MRI diffusion tomographic image MD₁ of the test subject 5. The image processing section 12 reconstructs an image from the tomographic images of the test subject 5 to acquire an axial transverse image (hereinafter referred to as the MRI diffusion axial transverse image) MD₂ of the test subject 5 as shown in, for example, FIG. 7.

The MRI device 7 simultaneously generates the static magnetic field and the gradient magnetic field in the casing 8 for generation of the magnetic field. In this state, the MRI device 7 generates the high-frequency magnetic field from the RF coil, and detects, via the RF coil, the weak resonance signal at a time when the high-frequency magnetic field is applied to the test subject 5 to output the MR signal. The image processing section 12 subjects the MR signal to the two-dimensional/three-dimensional Fourier transform to reconstruct an MRI morphological image. The MRI morphological image is a morphological image indicating a shape, a size, movement or the like of a bone, a tumor or the like of the test subject 5.

An apparatus main body 13 includes a computer constituted of a CPU, an ROM, an RAM and the like. The apparatus main body 13 fetches a plurality of different functional images, and superimposes these functional images on one another to generate a new functional image. Examples of the plurality of different functional images include the PET tomographic image PD₁ and the PET axial transverse image PD₂ acquired by the PET device 1. The plurality of different functional images include the MRI diffusion tomographic image MD₁ and the MRI diffusion axial transverse image MD₂ acquired by the MRI device 7.

Specifically, the apparatus main body 13 has a main control section 14 including the CPU and the like. The apparatus main body also has an image fetching section 15, a spatial resolution matching section 16, a correcting section 17, an alignment section 18, a subtracting section 19, a generating section 20, a count memory 21, a count incorporating section 22, a synthesis section 23, a displaying section 24 and a storage section 25 which operate in response to commands to be emitted from the main control section 14, respectively.

The image fetching section 15 fetches the PET tomographic image PD₁ or the PET axial transverse image PD₂ from the PET device 1 to store the PET tomographic image PD₁ or the PET axial transverse image PD₂ in the storage section 25. The image fetching section 15 fetches the MRI diffusion tomographic image MD₁ or the MRI diffusion axial transverse image MD₂ from the MRI device 7 to store the MRI diffusion tomographic image MD₁ or the MRI diffusion axial transverse image MD₂ in the storage section 25. The image fetching section 15 fetches the MRI morphological image from the MRI device 7 to store the image in the storage section 25.

The spatial resolution matching section 16 matches a spatial resolution of the PET tomographic image PD₁ acquired by the PET device 1 with that of the MRI diffusion tomographic image MD₁ acquired by the MRI device 7. The spatial resolution matching section 16 obtains, for example, the spatial resolutions of the PET tomographic image PD₁ and the MRI diffusion tomographic image MD₁, and matches the spatial resolution of the functional image other than the functional image having a low spatial resolution with the low spatial resolution. The spatial resolution matching section 16 matches, for example, the spatial resolution of the MRI diffusion tomographic image MD₁ with that of the PET tomographic image PD₁. The spatial resolution matching section 16 matches the spatial resolution of the MRI diffusion axial transverse image MD₂ with that of the PET axial transverse image PD₂. The spatial resolution matching section 16 uses, for example, a filter to reduce the spatial resolution as a technique of matching the spatial resolution.

The correcting section 17 corrects a strain of the MRI diffusion tomographic image MD₁ or the MRI diffusion axial transverse image MD₂ acquired by the MRI device 7. As described above, the phase of the MRI signal slightly changes with the micro-movements (diffusions) of the water molecules. In the diffusion imaging, the phase change of the MRI signal is captured and imaged by the imaging sequence referred to as a motion probing gradient (MPG) pulse. The MPG pulse is an additional gradient magnetic pulse to emphasize a phase deviation of transverse magnetization spin due to fine movements of molecules including target nuclear species of image pixels.

When an imaging object has a low speed, a gradient magnetic intensity of the additional gradient magnetic pulse needs to be increased independently of the gradient magnetic field required for the imaging. Especially for the MPG, the gradient magnetic intensity needs to be increased. The MPG of the maximum gradient magnetic intensity of an apparatus for use is applied for several tens of milliseconds in some case. When such an additional gradient magnetic pulse is applied, there is a problem that an unnecessary magnetic distribution occurs in the imaging during echo collection owing to an influence of adjustment deviation or the like of an eddy magnetic field compensation circuit which compensates for an eddy magnetic field generated by the application of the additional gradient magnetic pulse. There are reported a large number of cases where a change of a phase component, for example, the strain of the image or the like is caused by the generation of the unnecessary magnetic distribution.

A technique of suppressing the strain of the image or the like generated by the application of such an additional gradient magnetic pulse is disclosed in, for example, Jpn. Pat. No. 3,679,892 described above. It is disclosed in Jpn. Pat. No. 3,679,892 that a magnetic resonance imaging device to apply the additional gradient magnetic pulse in addition to the gradient magnetic field required for the imaging includes frequency change means for changing the central frequency of an echo signal wave during the imaging in order to correct deterioration of an image quality caused by the application of the additional gradient magnetic pulse. Therefore, the correcting section 17 corrects the strains of the MRI diffusion tomographic image MD₁ and the MRI diffusion axial transverse image MD₂ by use of the technique disclosed in, for example, Jpn. Pat. No. 3,679,892.

The alignment section 18 aligns the functional images having the spatial resolutions matched with one another by the spatial resolution matching section 16. The alignment section 18 aligns, for example, the PET tomographic image PD₁ and the MRI diffusion tomographic image MD₁. The alignment section 18 aligns, for example, the PET axial transverse image PD₂ and the MRI diffusion axial transverse image MD₂. To simplify description, the embodiment will be described in accordance with the PET axial transverse image PD₂ and the MRI diffusion axial transverse image MD₂.

The subtracting section 19 removes noise components included in the functional images aligned by the alignment section 18, respectively. The subtracting section 19 sets, for example, threshold values in order to remove the noise components included in the PET axial transverse image PD₂ and the MRI diffusion axial transverse image MD₂, respectively. The subtracting section 19 subtracts the threshold value from the PET axial transverse image PD₂, and also subtracts the threshold value from the MRI diffusion axial transverse image MD₂.

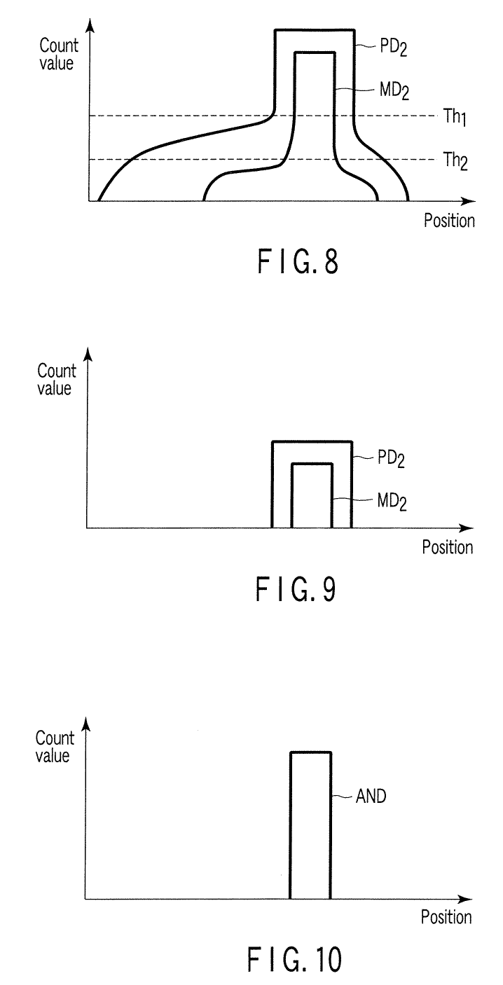

FIG. 8 shows examples of count values with respect to positions of the PET axial transverse image PD₂ and the MRI diffusion axial transverse image MD₂. The count value is a count value output at a time when the pair of detectors 2a and 2b, 3a and 3b, . . . , or n and nb simultaneously detect the gamma rays. The subtracting section 19 sets a threshold value Th₁ with respect to the PET axial transverse image PD₂, subtracts the threshold value Th₁ from the PET axial transverse image PD₂, and calculates the PET axial transverse image PD₂ shown in FIG. 9. Moreover, the subtracting section 19 sets a threshold value Th₂ with respect to the MRI diffusion axial transverse image MD₂, subtracts the threshold value Th₂ from the MRI diffusion axial transverse image MD₂, and calculates the MRI diffusion axial transverse image MD₂ shown in FIG. 9.

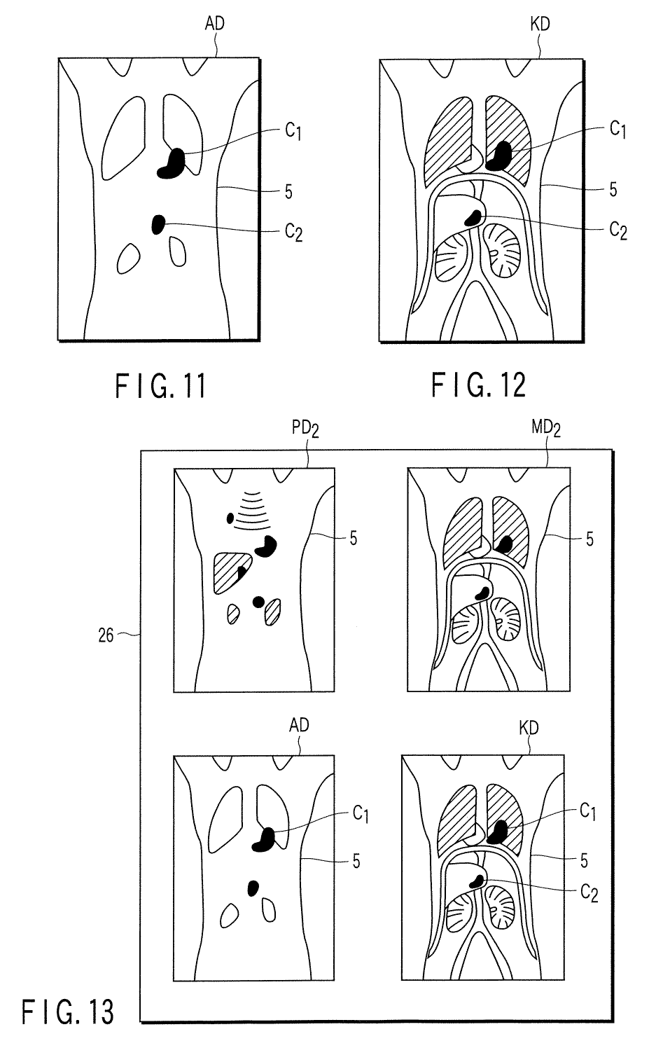

The generating section 20 takes a logical product (AND) of the functional images from which the threshold values have been subtracted by the subtracting section 19 to generate a new functional image (hereinafter referred to as the AND image). The generating section 20 takes a logical product (AND) of the PET axial transverse image PD₂ and the MRI diffusion axial transverse image MD₂ shown in, for example, FIG. 9 to generate an AND image shown in FIG. 10. FIG. 11 schematically shows an example of an AND image AD. There is a high probability that regions C₁, C₂ which appear in the AND image AD are estimated as cancers.

The new AND image AD generated by the generating section 20 includes a plurality of pixels. The count value itself of the AND image AD does not have any meaning, for example, when the cancers are estimated. When the pair of detectors 2a and 2b, 3a and 3b, . . . , or n and nb simultaneously detect the gamma rays, for example, the pair of detectors 2a, 2b output one count. The image processing section 6 fetches the count outputs of the pairs of detectors 2a and 2b, 3a and 3b, . . . , and n and nb to output the PET tomographic image of the test subject 5.

Therefore, an appropriately constant count value is buried in a pixel having a significant count value or more, for example, a count value corresponding to the estimation of the cancer or more among a plurality of pixels of the AND image AD. In consequence, the AND image AD of functional images such as the PET tomographic image PD₁ and the MRI diffusion tomographic image MD₁ is generated. For this purpose, the apparatus main body 13 has the count memory 21, the count incorporating section 22 and the synthesis section 23.

The count memory 21 stores a count value of a position signal generated during the detection of the gamma ray based on the radioactive marker administered to the test subject 5, for example, FDG as the nuclear species ¹⁸F in accordance with each pixel in, for example, the AND image AD of the PET axial transverse image PD₂ and the MRI diffusion axial transverse image MD₂.

The count incorporating section 22 incorporates the constant count value in the pixel of the AND image, when the count value stored in the count memory 21 is a preset threshold value or more.

The synthesis section 23 receives the AND image AD in which the constant count value has been incorporated by the count incorporating section 22, and superimposes, on this AND image AD, a morphological image of, for example, an MRI morphology acquired by the MRI device 7. The device capable of acquiring the morphological image is not limited to the MRI device 7. The synthesis section 23 may superimpose, on the AND image, the morphological image acquired by a medical image device capable of acquiring the morphological image. Examples of the medical image device capable of acquiring the morphological image include an X-ray computer tomography imaging device, an X-ray device and an ultrasonic diagnosis device. FIG. 12 shows one example of an image KD obtained by superimposing the AND image AD on an MRI morphological image acquired by the MRI device 7.

The displaying section 24 displays the AND image AD generated by the generating section 20 in a display screen of a display 26. As shown in, for example, FIG. 13, the displaying section 24 arranges and displays the AND image AD, the PET axial transverse image PD₂ acquired by the PET device 1, and the MRI diffusion axial transverse image MD₂ acquired by the MRI device 7. The displaying section 24 arranges and displays the superimposed image KD acquired by the synthesis section 23 which superimposes the AND image AD on the MRI morphological image acquired by the MRI device 7.

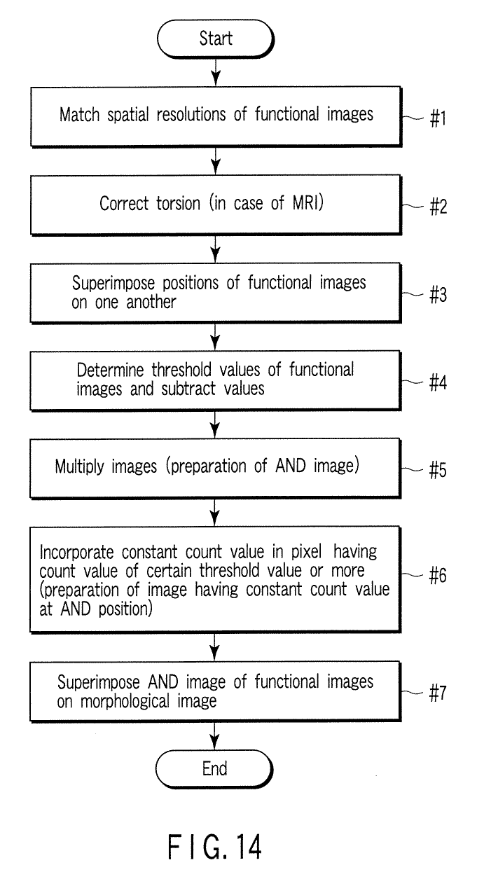

Next, medical image synthesis by the apparatus constituted as described above will be described with reference to a medical image synthesis flow chart shown in FIG. 14.

The radioactive FDG marker, for example, the nuclear species ¹⁸F is administered into the human body as the test subject. The PET device 1 tracks the behavior at a time when the radioactive FDG marker, for example, the nuclear species ¹⁸F is administered into the human body as the test subject. The device outputs the functional image in which the cancerous region is imaged by use of, for example, the fact that more glucose is taken in the cancerous region than in the normal region, for example, the PET tomographic image PD₁ of the test subject 5 shown in FIG. 3. When the whole test subject 5 is scanned by the PET device 1, the image processing section 6 reconstructs the image from the tomographic images of the test subject 5 to output the PET axial transverse image PD₂ shown in FIG. 4.

On the other hand, the MRI device 7 acquires the MRI diffusion tomographic image MD₁ of the test subject 5 by the diffusion imaging as shown in, for example, FIG. 6. The MRI device 7 reconstructs the image from the tomographic images of the test subject 5 to acquire the MRI diffusion axial transverse image MD₂ as shown in, for example, FIG. 7. The MRI device 7 also acquires the MRI morphological image as the morphological image indicating the shape, the size, the movement or the like of the bone, the tumor or the like of the test subject 5.

Subsequently, the image fetching section 15 fetches the PET axial transverse image PD₂ from, for example, the PET device 1 to store the image in the storage section 25. The image fetching section 15 fetches the MRI diffusion axial transverse image MD₂ from the MRI device 7 to store the image in the storage section 25. Alternatively, the image fetching section 15 fetches the MRI image as the morphological image from the MRI device 7 to store the image in the storage section 25.

Next, in step #1, the spatial resolution matching section 16 obtains the spatial resolutions of the PET axial transverse image PD₂ acquired by the PET device 1 and the MRI diffusion axial transverse image MD₂ acquired by the MRI device 7. The spatial resolution matching section 16 matches the spatial resolution of the functional image other than the functional image having the low spatial resolution with the low spatial resolution. For example, the spatial resolution matching section matches the spatial resolution of the MRI diffusion axial transverse image MD₂ with that of the PET axial transverse image PD₂. In this case, in the spatial resolution matching section 16, for example, the filter which reduces the spatial resolution is used as the technique of matching the spatial resolution.

Subsequently, in step #2, the correcting section 17 corrects the strain of the MRI diffusion axial transverse image MD₂ acquired by the MRI device 7. This strain is corrected in order to suppress the strain of the image or the like caused by the application of the additional gradient magnetic pulse in the MRI device 7.

Subsequently, in step #3, the alignment section 18 aligns the PET axial transverse image PD₂ and the MRI diffusion axial transverse image MD₂ having the spatial resolutions matched with each other by the spatial resolution matching section 16.

Subsequently, in step #4, the subtracting section 19 sets the threshold values in order to remove the noise components included in the PET axial transverse image PD₂ and the MRI diffusion axial transverse image MD₂ aligned by the alignment section 18, respectively. In the subtracting section 19, for example, as shown by the count values during the simultaneous detection of the gamma rays with respect to the positions of the PET axial transverse image PD₂ and the MRI diffusion axial transverse image MD₂ in FIG. 8, the threshold value Th₁ is set with respect to the PET axial transverse image PD₂, and the threshold value Th₂ is set with respect to the MRI diffusion axial transverse image MD₂.

The subtracting section 19 subtracts the threshold value Th₁ from the PET axial transverse image PD₂ to calculate the PET axial transverse image PD₂ shown in FIG. 9. Moreover, the subtracting section 19 subtracts the threshold value Th₂ from the MRI diffusion axial transverse image MD₂ to calculate the MRI diffusion axial transverse image MD₂ shown in FIG. 9.

Subsequently, in step #5, the generating section 20 takes the logical product (AND) of the PET axial transverse image PD₂ and the MRI diffusion axial transverse image MD₂ calculated by the subtracting section 19 as shown in FIG. 9 to generate the AND image AD shown in FIG. 10. FIG. 11 schematically shows the example of the AND image AD. The regions C₁, C₂ which appear in the AND image AD indicate large count values shown in FIG. 10, and there is a high probability that the regions are estimated as the cancers. At this time, the displaying section 24 receives the AND image AD generated by the generating section 20, and displays the AND image AD in the display screen of the display 26.

Specifically, the appropriately constant count value is buried in the pixel having the significant count value or more, for example, the count value corresponding to the estimation of the cancer or more among the plurality of pixels of the AND image AD. In consequence, the AND image AD of functional images such as the PET tomographic image PD₁ and the MRI diffusion tomographic image MD₁ is generated.

That is, the count memory 21 stores the count value of the position signal generated during the detection of the gamma ray based on a radioactive marker such as FDG administered to the test subject 5, for example, the nuclear species ¹⁸F in accordance with each pixel in, for example, the AND image AD of the PET axial transverse image PD₂ and the MRI diffusion axial transverse image MD₂.

In step #6, the count incorporating section 22 incorporates the constant count value in the pixel of the AND image AD, when the count value stored in the count memory 21 is the preset threshold value or more. In consequence, since the constant count value is incorporated in the pixel having the preset threshold value or more in the AND image AD, for example, the cancerous region having the preset threshold value or more forms an emphasized image having an increased count value.

Subsequently, in step #7, the synthesis section 23 receives the AND image AD in which the constant count value has been incorporated by the count incorporating section 22, and superimposes, on this AND image AD, a morphological image such as the MRI morphological image acquired by the MRI device 7. The synthesis section 23 superimposes the AND image AD on the MRI morphological image acquired by the MRI device 7 to acquire the superimposed image KD as shown in FIG. 12.

The displaying section 24 displays the AND image AD generated by the generating section 20 in the display screen of the display 26. As shown in, for example, FIG. 13, the displaying section 24 arranges and displays the AND image AD, the PET axial transverse image PD₂ acquired by the PET device 1, and the MRI diffusion axial transverse image MD₂ acquired by the MRI device 7. The displaying section 24 arranges and displays the superimposed image KD acquired by the synthesis section 23 which superimposes the AND image AD on the MRI morphological image acquired by the MRI device 7.

As described above, according to the first embodiment, the AND image AD of the PET axial transverse image PD₂ acquired by the PET device 1 and the MRI diffusion axial transverse image MD₂ acquired by the MRI device 7 is generated, and displayed in the display screen of the display 26. Here, the PET axial transverse image PD₂ and the MRI diffusion axial transverse image MD₂ have the low probability that the cancerous regions are indicated, and the images indicate whether or not the cancerous regions are present. Even when the PET axial transverse image PD₂ and the MRI diffusion axial transverse image MD₂ are such images, the AND image AD of the axial transverse images is acquired. In consequence, the regions C₁, C₂ which appear on the AND image AD are emphasized and have a high probability that the regions are estimated as the cancers.

The cancerous regions C₁, C₂ must be reflected on the PET axial transverse image PD₂ and the MRI diffusion axial transverse image MD₂, respectively. Moreover, a region other than the cancerous region is not reflected on both of the PET axial transverse image PD₂ and the MRI diffusion axial transverse image MD₂, and must be reflected on one of the images.

When the AND image AD of the PET axial transverse image PD₂ and the MRI diffusion axial transverse image MD₂ is acquired, the regions C₁, C₂ reflected on the AND image AD have a very high possibility of the cancer. Therefore, the cancerous regions C₁, C₂ can clearly be reflected on the image with high probability as compared with a single functional image such as an image simply generated by superimposing a morphological image such as the MRI morphological image on the PET axial transverse image PD₂.

The count memory 21 stores the count value of the position signal generated during the detection of the gamma ray based on a radioactive marker such as FDG administered to the test subject 5, for example, the nuclear species ¹⁸F in accordance with each pixel in, for example, the AND image AD of the PET axial transverse image PD₂ and the MRI diffusion axial transverse image MD₂. The count incorporating section 22 incorporates the constant count value in the pixel of the AND image, when the count value stored in the count memory 21 is the preset threshold value or more. In consequence, for example, in the cancerous region having the preset threshold value or more, the emphasized image having an increased count value is formed. The probability that the regions C₁, C₂ are estimated as the cancers can further increase.

The displaying section 24 displays the following images in the display screen of the display 26. That is, the displaying section 24 displays the AND image AD generated by the generating section 20. As shown in, for example, FIG. 13, the displaying section 24 arranges and displays the AND image AD, the PET axial transverse image PD₂ acquired by the PET device 1, and the MRI diffusion axial transverse image MD₂ acquired by the MRI device 7. The displaying section 24 arranges and displays the superimposed image KD acquired by the synthesis section 23. In consequence, since the images displayed in the display screen of the display 26 are observed, the regions C₁, C₂ can be specified as places of the cancerous regions.

From the MRI morphological image acquired by the MRI device 7, the shape, the size, the movement or the like of the bone, the tumor or the like of the human body as the test subject 5 can be seen. Therefore, when the superimposed image KD of the AND image AD and the MRI morphological image is displayed, the places of the regions C₁, C₂ can easily be specified as the cancerous regions.

It is to be noted that, to simplify the description, the first embodiment has been described using the PET axial transverse image PD₂ and the MRI diffusion axial transverse image MD₂. However, the present invention is not limited to this embodiment. Needless to say, an AND image of the PET tomographic image PD₁ acquired by the PET device 1 and the MRI diffusion tomographic image MD₁ acquired by the MRI device 7 may be obtained.

This embodiment will briefly be described. In the step #1, the spatial resolution matching section 16 matches the spatial resolution of the PET tomographic image PD₁ with that of the MRI diffusion tomographic image MD₁.

Subsequently, in the step #2, the correcting section 17 corrects the strain of the MRI diffusion tomographic image MD₁.

Subsequently, in the step #3, the alignment section 18 aligns the PET tomographic image PD₁ and the MRI diffusion tomographic image MD₁ having the spatial resolutions matched with each other by the spatial resolution matching section 16.

Subsequently, in the step #4, the subtracting section 19 sets the threshold values in order to remove the noise components included in the PET tomographic image PD₁ and the MRI diffusion tomographic image MD₁ aligned by the alignment section 18, respectively, and subtracts the values.

Subsequently, in the step #5, the generating section 20 takes a logical product (AND) of the PET tomographic image PD₁ and the MRI diffusion tomographic image MD₁ calculated by the subtracting section 19 to generate an AND image. This AND image AD is displayed in the display screen of the display 26.

The count memory 21 stores the count value of the position signal generated during the detection of the gamma ray based on the radioactive marker administered to the test subject 5 in accordance with each pixel in, for example, the AND image AD of the PET tomographic image PD₁ and the MRI diffusion tomographic image MD₁.

In the step #6, the count incorporating section 22 incorporates the constant count value in the pixel of the AND image AD, when the count value stored in the count memory 21 is a preset threshold value or more. In consequence, since the constant count value is incorporated in the pixel having the preset threshold value or more in the AND image AD, for example, the cancerous region having the preset threshold value or more indicates the increased count value to form the emphasized image.

Subsequently, in the step #7, the synthesis section 23 receives the AND image AD in which the constant count value has been incorporated by the count incorporating section 22, and superimposes, on this AND image AD, a morphological image such as the MRI morphological image acquired by the MRI device 7 to acquire the superimposed image.

The displaying section 24 displays the AND image AD generated by the generating section 20 in the display screen of the display 26. The displaying section 24 arranges and displays the AND image, the PET tomographic image PD₁ and the MRI diffusion tomographic image MD₁. The displaying section 24 arranges and displays the superimposed image acquired by the synthesis section 23.

Next, another embodiment will be described.

In the first embodiment, the PET device 1 as the medical image device to acquire the functional image, and the diffusion imaging of the MRI device 7 have been described. The present invention is not limited to this embodiment. A SPECT device or a US device may be used.

The present invention is not limited to the AND image of the PET axial transverse image PD₂ and the MRI diffusion axial transverse image MD₂. An AND image of a plurality of functional images may be generated. For example, an AND image of at least two functional images of the PET axial transverse image PD₂, the MRI diffusion axial transverse image MD₂, a functional image acquired by the SPECT device and a functional US image acquired by the US device may be generated.

To generate the AND image, for example, an AND image of a plurality of PET tomographic images PD₁ or a plurality of PET axial transverse images PD₂ acquired by the PET device 1 may be generated. In this case, the AND image of the plurality of PET tomographic images PD₁ or the plurality of PET axial transverse images PD₂ having different imaging times may be generated.

In the first embodiment, the AND image AD of the PET axial transverse image PD₂ and the MRI diffusion axial transverse image MD₂ is generated, and the morphological image acquired by the MRI device 7 is superimposed on this AND image AD to generate the image KD, but the functional image acquired by the SPECT device or the US device may be superimposed on the AND image AD.

A medical image device to acquire a morphological image is not limited to the MRI device 7, and an X-ray CT device, an X-ray device or the US device may be used.

In the first embodiment, it has been described above that the AND image of a PET image and the MRI diffusion image is generated, and the MRI morphological image is superimposed on this AND image to generate a composite image. To generate the composite image, formed images acquired as follows may be superimposed. For example, the composite image is generated using an image of a deviation between the PET image and the MRI morphological image. The image is generated using an image of a deviation between the MRI diffusion image and the MRI morphological image. The image is generated using an image of a deviation between the PET image and the MRI diffusion image. An AND image of the PET image and the MRI diffusion image is generated, and the image is generated using an image of a deviation between this AND image and the MRI morphological image. An AND image of the PET image and the MRI diffusion image is generated, and the image is generated using an image of a deviation between this AND image and the PET image. An AND image of the PET image and the MRI diffusion image is generated, and the image is generated using an image of a deviation between this AND image and the MRI diffusion image.

The preparation of the AND image is not limited to that of the AND image of two functional images such as the PET image and the MRI diffusion image. The AND image may be prepared using at least three functional images such as a first PET image, a second PET image and the MRI diffusion image. In this case, the first PET image is different from the second PET image in, for example, a type or a collection date of a radioactive marker administered to the test subject 5.

Next, a second embodiment of the present invention will be described. It is to be noted that a respect different from that of the first embodiment will be described.

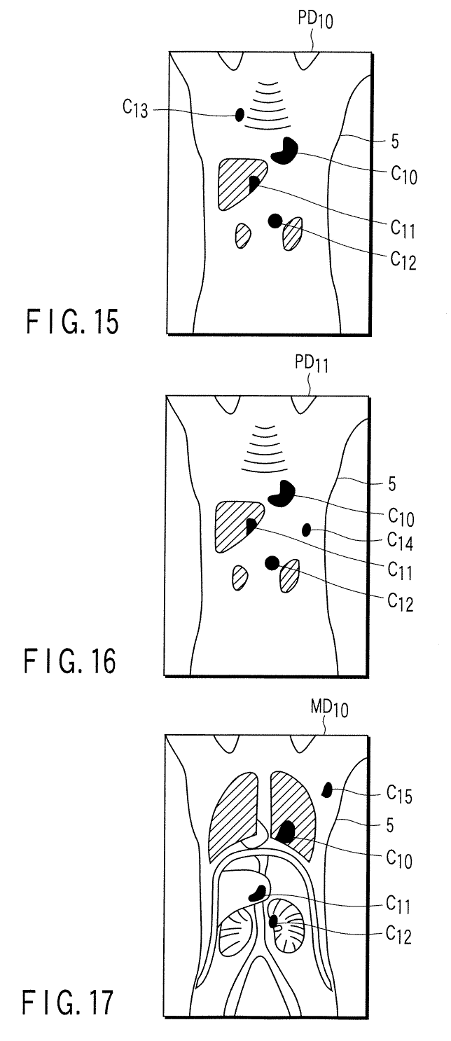

An apparatus main body 13 acquires at least three different functional images in which functions of a test subject are imaged, and takes images having a preset ratio or more of these functional images to generate a new functional image. That is, an image fetching section 15 fetches at least three different functional images in which the functions of the test subject are imaged. For example, the image fetching section 15 fetches, for example, a first PET image PD₁₀ shown in FIG. 15 and a second PET image PD₁₁ shown in FIG. 16 from a PET device 1. The first PET image PD₁₀ is different from the second PET image PD₁₁ in, for example, the type of the radioactive marker administered to a test subject 5. The image fetching section 15 fetches, for example, an MRI diffusion image MD₁₀ shown in FIG. 17 from an MRI device 7.

When a ratio having a predetermined pixel value in the same part of the first PET image PD₁₀, the second PET image PD₁₁ and the MRI diffusion image MD₁₀ is a preset ratio or more, the apparatus main body 13 takes the part of the pixel value to generate a new functional image.

This respect will specifically be described. Each of the first PET image PD₁₀, the second PET image PD₁₁ and the MRI diffusion image MD₁₀ includes a plurality of pixels.

A spatial resolution matching section 16 matches spatial resolutions of the first PET image PD₁₀, the second PET image PD₁₁ and the MRI diffusion image MD₁₀ with one another.

Subsequently, a correcting section 17 corrects a strain of the MRI diffusion image MD₁₀.

Subsequently, an alignment section 18 aligns the first PET image PD₁₀, the second PET image PD₁₁ and the MRI diffusion image MD₁₀ having the spatial resolutions matched with one another by the spatial resolution matching section 16.

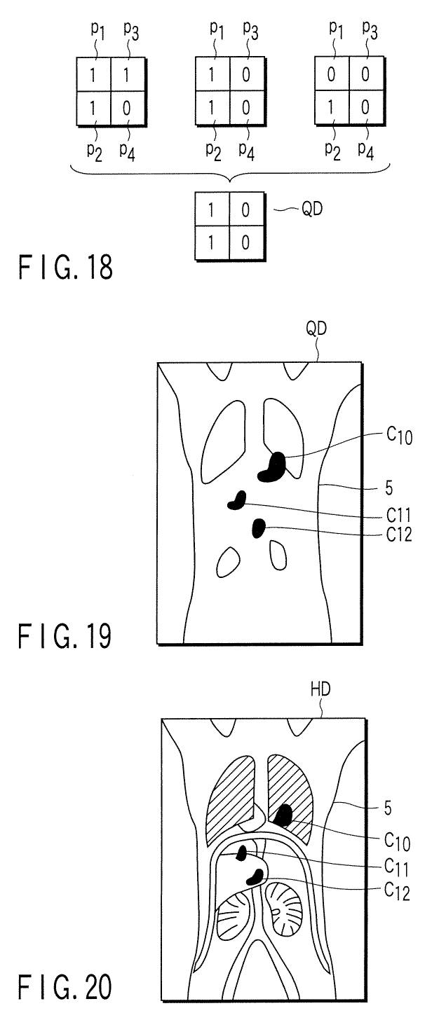

Subsequently, when the ratio having the predetermined pixel value in the same part of the first PET image PD₁₀, the second PET image PD₁₁ and the MRI diffusion image MD₁₀ is the preset ratio or more, a synthesis section 23 takes the part of the pixel value to generate the new functional image. That is, FIG. 18 shows count values of pixels of an n×n matrix in the same part of the first PET image PD₁₀, the second PET image PD₁₁ and the MRI diffusion image MD₁₀. The values will hereinafter be referred to simply as the count values of the n×n pixels. In this case, n is a positive integer. The drawing shows the count values of 2×2 pixels in order to simplify the description. The pixels have portions, for example, p₁ to p₄.

The synthesis section 23 takes the pixel as “1” to generate a new functional image QD, when, for example, the pixels of two of the three images including the first PET image PD₁₀, the second PET image PD₁₁ and the MRI diffusion image MD₁₀ have a count value “1”. That is, when the count value “1” is indicated at a ratio of ⅔ or more among the three images, the pixel is taken as “1” to generate the new functional image QD. This ratio is not limited to ⅔, and can arbitrarily be set.

Therefore, when the ratio having the predetermined pixel value “1” is set to ⅔ and when, as shown in, for example, FIG. 18, the pixel p₁ of the first PET image PD₁₀ is “1”, the pixel p₁ of the second PET image PD₁₁ is “1” and the pixel p₁ of the MRI diffusion image MD₁₀ is “0”, the synthesis section 23 sets the pixel p₁ as “1” to generate the new functional image QD. When the pixel p₂ of the first PET image PD₁₀ is “1”, the pixel p₂ of the second PET image PD₁₁ is “1” and the pixel p₂ of the MRI diffusion image MD₁₀ is “1”, the synthesis section 23 sets the pixel p₂ as “1” to generate the new functional image QD. Hereinafter, similarly, the synthesis section 23 sets the pixel p₃ as “0” to generate the new functional image QD, and sets the pixel p4 as “0” to generate the new functional image QD.

FIG. 19 shows the whole schematic view of the new functional image QD. In the new functional image QD, regions C₁₀, C₁₁ and C₁₂ appear in which the count values of the pixels of, for example, two of three images including the first PET image PD₁₀, the second PET image PD₁₁ and the MRI diffusion image MD₁₀ are A count incorporating section 22 incorporates a constant count value in, for example, the pixels p₁, p₂ having the count value “1”. In consequence, the regions C₁₀, C₁₁ and C₁₂ estimated as, for example, cancerous regions form emphasized images having the increased count value.

Subsequently, the synthesis section 23 superimposes, on the new functional image QD, a morphological image such as the MRI morphological image acquired by the MRI device 7 to acquire a superimposed image HD. FIG. 20 shows the superimposed image HD obtained by superimposing the MRI morphological image on the new functional image QD.

A displaying section 24 displays the new functional image QD generated by the generating section 20 in the display screen of the display 26. The displaying section 24 arranges and displays the new functional image QD, a PET tomographic image PD₁ and an MRI diffusion tomographic image MD₁. The displaying section 24 displays the superimposed image HD obtained by superimposing, on the new functional image QD, a morphological image such as the MRI morphological image acquired by the MRI device 7.

As described above, according to the second embodiment, when the ratio having the predetermined pixel value “1” in the same part of the first PET image PD₁₀, the second PET image PD₁₁ and the MRI diffusion image MD₁₀ is a preset ratio of ⅔ or more, the part of the pixel value is taken to generate the new functional image QD. Therefore, needless to say, an effect similar to that of the first embodiment is produced.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical image synthesis method comprising:
acquiring a plurality of different functional images in which functions of a test subject are imaged;
taking a logical product of the plurality of different functional images to generate new information; and
superimposing and displaying the new information on a corresponding position of the functional image of the test subject.

2. A medical image synthesis method comprising:
acquiring a plurality of different functional images in which functions of a test subject are imaged; and
taking a logical product of the plurality of different functional images to generate a new functional image.

3. The medical image synthesis method according to claim 2, wherein:
spatial resolutions of the plurality of different functional images are matched with one another, and
the logical product of the plurality of functional images having the spatial resolutions matched with one another is taken to generate the new functional image.

4. The medical image synthesis method according to claim 2, wherein the spatial resolutions of the functional images other than the functional image having the lowest spatial resolution among the plurality of functional images are matched with the lowest spatial resolution.

5. The medical image synthesis method according to claim 1 or 2, wherein the plurality of different functional images are acquired by any one of medical image devices of a magnetic resonance imaging device, a positron emission computed tomography imaging device, a single photon emission computed tomography imaging device and an ultrasonic diagnosis device.

6. The medical image synthesis method according to claim 1 or 2, wherein the plurality of different functional images include an image acquired by a nuclear medicine device and an image acquired by magnetic resonance imaging.

7. The medical image synthesis method according to claim 6, wherein the image acquired by the magnetic resonance imaging includes a diffusion image.

8. The medical image synthesis method according to claim 1 or 2, wherein the plurality of different functional images include an image acquired by positron emission computed tomography imaging and an image acquired by single photon emission computed tomography imaging.

9. The medical image synthesis method according to claim 1 or 2, wherein the plurality of different functional images are acquired by a nuclear medicine device, respectively, and radioactive markers to be administered to the test subject are different.

10. The medical image synthesis method according to claim 1 or 2, wherein the plurality of different functional images are acquired by the same medical image device, respectively, and times when the images have been acquired are different.

11. The medical image synthesis method according to claim 2, further comprising:
synthesizing the new functional image with a morphological image in which at least a shape of the test subject is imaged.

12. A medical image synthesis method comprising:
using, as a plurality of different functional images, a functional image acquired by positron emission computed tomography imaging and a diffusion image acquired by magnetic resonance imaging;
matching a spatial resolution of the diffusion image with that of the functional image;
correcting a strain of the diffusion image;
aligning the functional image having the matched spatial resolution and the diffusion image having the corrected strain; and
taking a logical product of the functional image and the diffusion image which have been aligned to generate a new functional image.

13. The medical image synthesis method according to claim 12, wherein:
the new functional image includes a plurality of pixels,
a count value of a position signal generated during detection of a gamma ray based on a radioactive marker to be administered to the test subject in accordance with the plurality of pixels is stored, and
a constant count value is stored in the pixel, when the count value is a preset threshold value or more.

14. A medical image synthesis method comprising:
acquiring at least three different functional images in which functions of a test subject are imaged; and
generating a new functional image based on image parts having a preset ratio or more of the at least three different functional images.

15. The medical image synthesis method according to claim 14, wherein when a ratio having a predetermined pixel value in the same part of the at least three different functional images is the preset ratio or more, the new functional image is generated based on parts of the pixel value having the preset ratio or more.

16. The medical image synthesis method according to claim 14, further comprising:
synthesizing the new functional image with a morphological image in which at least a shape of the test subject is imaged.

17. A medical image synthesis apparatus comprising:
an image fetching section which fetches a plurality of different functional images acquired by a plurality of medical image devices, respectively;
a generating section which takes a logical product of the plurality of different functional images to generate new information;
a display; and
a displaying section which superimposes the new information on a corresponding position of the functional image of a test subject to display the information in the display.

18. A medical image synthesis apparatus comprising:
an image generation unit which fetches a plurality of different functional images acquired by a plurality of medical image devices, respectively, and which takes a logical product of the plurality of different functional images to generate a new functional image.

19. The medical image synthesis apparatus according to claim 18, wherein:
the image generation unit includes a spatial resolution matching section which matches spatial resolutions of the plurality of different functional images with one another, and
a generating section which takes the logical product of the plurality of functional images having the spatial resolutions matched with one another to generate a new functional image.

20. The medical image synthesis apparatus according to claim 18, wherein the spatial resolution matching section matches the spatial resolutions of the functional images other than the functional image having the lowest spatial resolution among the plurality of functional images with the lowest spatial resolution.

21. The medical image synthesis apparatus according to claim 17 or 18, wherein the plurality of medical image devices include a magnetic resonance imaging device, a positron emission computed tomography imaging device, a single photon emission computed tomography imaging device and an ultrasonic diagnosis device.

22. The medical image synthesis apparatus according to claim 18, wherein:
the plurality of medical image devices are a nuclear medicine device and a magnetic resonance imaging device, and
the image generation unit fetches an image acquired by the nuclear medicine device and an image acquired by the magnetic resonance imaging device, and takes a logical product of these images to generate the new functional image.

23. The medical image synthesis apparatus according to claim 22, wherein:
the magnetic resonance imaging device acquires a diffusion image as the image.

24. The medical image synthesis apparatus according to claim 18, wherein:
the plurality of medical image devices are a positron emission computed tomography imaging device and a single photon emission computed tomography imaging device, and
the image generation unit fetches an image acquired by the positron emission computed tomography imaging device and an image acquired by the single photon emission computed tomography imaging device, and takes a logical product of these images to generate the new functional image.

25. The medical image synthesis apparatus according to claim 17 or 18, wherein:
the medical image device is a nuclear medicine device, and
the image generation unit fetches the plurality of different functional images acquired by the nuclear medicine device when different radioactive markers are administered to the test subjects, respectively, and takes a logical product of these images to generate the new functional image.

26. The medical image synthesis apparatus according to claim 17 or 18, wherein the medical image devices acquire the plurality of functional images of the test subject at different times, respectively.

27. The medical image synthesis apparatus according to claim 18, wherein the image generation unit includes a synthesis section which synthesizes the new functional image with a morphological image where at least a shape of the test subject is imaged.

28. A medical image synthesis apparatus comprising:
an image acquiring section which fetches a functional image acquired by a positron emission computed tomography imaging device and a diffusion image acquired by a magnetic resonance imaging device;
a spatial resolution matching section which matches a spatial resolution of the diffusion image with that of the functional image;
a correcting section which corrects a strain of the diffusion image;
an alignment section which aligns the functional image having the spatial resolution matched by the spatial resolution matching section and the diffusion image having the corrected strain; and
a generating section which takes a logical product of the functional image and the diffusion image which have been aligned by the alignment section to generate a new functional image.

29. The medical image synthesis apparatus according to claim 28, wherein:
the new functional image includes a plurality of pixels,
the medical image synthesis apparatus further comprising:
a count memory which stores a count value of a position signal generated during detection of a gamma ray based on a radioactive marker administered to the test subject in accordance with the plurality of pixels; and
a count incorporating section which incorporates a constant count value in the pixel, when the count value stored in the count memory is a preset threshold value or more.

30. A medical image synthesis apparatus comprising:
an image generation unit which acquires at least three different functional images where functions of a test subject are imaged and which generates a new functional image based on image parts having a preset ratio or more of the at least three different functional images.

31. The medical image synthesis apparatus according to claim 30, wherein when a ratio having a predetermined pixel value in the same part of the at least three different functional images having a predetermined pixel value is the preset ratio or more, the image generation unit generates the new functional image based on the parts of the pixel value having the preset ratio or more.

32. The medical image synthesis apparatus according to claim 30, wherein the image generation unit includes a synthesis section which synthesizes the new functional image with a morphological image in which at least a shape of the test subject is imaged.
